(19) United States
(12) Patent Application Publication
(10) Pub. No.: US 2017/0010280 A1
(43) Pub. Date: Jan. 12, 2017

(54) GDF15 AS BIOMARKER FOR DIAGNOSING MITOCHONDRIAL DISEASES

(71) Applicants: TOKYO METROPOLITAN GERIATRIC HOSPITAL AND INSTITUTE OF GERONTOLOGY, Tokyo (JP); KURUME UNIVERSITY, Kurume-shi (JP)

(72) Inventors: Masashi TANAKA, Tokyo (JP); Masafumi ITO, Tokyo (JP); Yasunori FUJITA, Tokyo (JP); Yasutoshi KOGA, Kurume-shi (JP)

(21) Appl. No.: 15/111,513

(22) PCT Filed: Jan. 14, 2015
(86) PCT No.: PCT/JP2015/050833

§ 371 Date: Sep. 26, 2016

(30) Foreign Application Priority Data
Jan. 15, 2014 (JP) 2014-005391
Oct. 31, 2014 (JP) 2014-223500

(51) Int. Cl. G01N 33/68; G01N 33/74; C12Q 1/68

(52) U.S. Cl. CPC G01N 33/6893; C12Q 1/6883; G01N 33/74; G01N 33/6863; C12Q 2600/158; G01N 2333/4753; G01N 2333/475; G01N 2800/04; G01N 2333/52

(57) ABSTRACT

To obtain data associated with a mitochondrial disease, a method includes measuring the level of at least one protein selected from the group consisting of GDF15 (growth differentiation factor 15), HGF (hepatocyte growth factor), MIG (gamma interferon induction monokine), SCF (stem cell factor) and SCGF-β (stem cell growth factor beta) in a biological sample collected from a subject. The measured protein level is compared to that of control subjects and then it is checked whether or not there is difference between the protein level of the subject and that of control subjects.

### TECHNICAL FIELD

The invention relates to GDF15 as biomarker for diagnosing mitochondrial diseases.

### BACKGROUND ART

A mitochondrion is a subcellular organelle in an eukaryotic cell. It produces ATP, which is used as energy in vivo, through the electron transfer system. If the energy producing capacity of mitochondria reduces, people may develop mitochondrial diseases. People with mitochondrial diseases can have damages in brain, skeletal muscle, cardiac muscle and other organs which need much energy.

The inventors have been studying mitochondria and mitochondrial diseases, and reported the results in patent applications (e.g., patent document 1) and journals (e.g., non-patent document 1). In non-patent document 1, we carried out a metabolome analysis of 2SA cell (control cell) and 2SD cell (mitochondrial disease model cell) and disclosed the effect of pyruvate administration for the energy metabolism of mitochondrial diseases model cell. In the document, we disclosed that the energy metabolism disorder has become remarkable 4 hours after treatment of high concentration of lactic acid (10 mM) in 2SD cells, and the phenomenon was not observed after treatment of high concentration of pyruvic acid (10 mM). The energy metabolism disorder was not observed after treatment of high concentration of lactic acid in 2SA cells.

### PRIOR ART DOCUMENTS

### Patent Document

Patent Document 1: JP 2007-330151 A

### Non-patent Document

Non-patent document 1: Kami K. et at, Metabolomic profiling rationalized pyruvate efficacy in cybrid cells harboring MELAS mitochondrial DNA mutations: Mitochondrion, 2012, 12(6), p 644-653

### SUMMARY OF THE INVENTION

### Problems to be Solved by the Invention

However, proteins for diagnosing mitochondrial diseases have not been fully known.

The present invention has been made in view of the problems described above, and its object is to provide molecules used as diagnostic biomarkers for mitochondrial diseases.

### Means for Solving the Problems

The first invention for solving the problems is a measuring method that obtains data associated with a mitochondrial disease comprising: measuring the level of at least one protein selected from the group consisting of GDF15 (growth differentiation factor 15), HGF (hepatocyte growth factor), MIG (gamma interferon induction monokine), SCF (stem cell factor) and SCGF-β (stem cell growth factor beta) in a biological sample collected from a subject; comparing the protein level to that of control subjects; and checking whether or not there is difference between the protein level of the subject and that of control subjects.

In the invention, it is preferable that checking the level of GDF15, HGF, MIG and SCF in the biological sample collected from the subject is higher than that in the biological sample collected from control subjects; and checking the level of SCGF-β in the biological sample collected from the subject is lower than that in the biological sample collected from control subjects.

The second invention is a measuring method about mitochondrial disease comprising: measuring the level of mRNA of at least one protein selected from the group consisting of GDF15 (growth differentiation factor 15), HGF (hepatocyte growth factor), MIG (gamma interferon induction monokine), SCF (stem cell factor) and SCGF-β (stem cell growth factor beta) in a biological sample collected from a subject; comparing the mRNA level to that of control subjects; and checking whether or not there is difference between the mRNA level of the subject and that of control subjects.

In the invention, it is preferable that the biological sample is blood.

Mitochondrial disease develop by decreasing aerobic energy production which mitochondrial mutations cause. Mitochondria contains own DNA (16569 base pairs in humans) apart from nuclear DNA. Molecules for the mitochondrial energy production is also encoded in nuclear DNA, in addition to the mitochondrial DNA. Therefore, mitochondrial diseases may be occurred by mutation of the regulation of nuclear DNA and proteins in addition to the mitochondrial DNA mutations. In mitochondrial disease patients, mitochondria is heteroplasmy, all of mitochondria is not always abnormal. Thus, mitochondrial diseases are known to exhibit a variety of disease states.

As mitochondrial disease, for example, chronic progressive external ophthalmoplegia syndrome (chronic progressive external ophthalmoplegia: CPEO), MELAS (mitochondrial myopathy, encephalopathy, lactic acidosis and stroke-like episodes), MERRF (myoclonus epilepsy associated with ragged-red fibers) and the like are known. In the invention, it can be directed to any of mitochondrial disease, especially m.3243A>G mutation (e.g., MELAS) is preferable.

GDF15 is a protein belonging to the TGF-β superfamily which have a function of regulating inflammation and apoptosis during progress of damaged tissue or disease. GDF15 is also known as TGF-PL, MIC-1, PDF, PLAB and PTGFB. However, the relationship between mitochondrial disease and GDF15 has not been known before. Further, HGF, MIG, SCF and SCGF-β, has not been known about the relationship between mitochondrial disease.

The other invention is a kit for carrying out the said method, comprising an antibody which specifically recognizes a protein selected from the group consisting of GDF15, HGF, MIG, SCF and SCGF-β. For the kit comprising an antibody, it is preferably used ELISA method.

Another invention is a kit for carrying out the said method, comprising DNA which recognizes mRNA that expresses the protein selected from the group consisting of GDF15, HGF, MIG, SCF and SCGF-β. For the kit comprising DNA, it is preferably used PCR method.

Another invention is a therapeutic kit comprising the said kit and therapeutic agent for a mitochondrial disease. Therapeutic agent for a mitochondrial disease contains sodium pyruvate, coenzyme Q10 or coenzyme Q10 analogue (Idebenone), EPI-743 and L-arginine. By administering mitochondrial disease therapeutic agent, it may be recognized therapeutic effect of mitochondrial disease patients. Therefore, by combining the measurement kit and mitochondrial disease therapeutic agent, the therapeutic agent is administered with the therapeutic effect. The invention is preferably used.

### Effects of the Invention

According to the invention, GDF15, HGF, MIG, SCF and SCGF-β are used for diagnostic biomarkers of mitochondrial disease. That is, the blood levels of GDF15, HGF, MIG, SCF and SCGF-β in the mitochondrial disease patients are different from those in the control subjects (the levels of GDF15, HGF, MIG and SCF is higher and the level of SCGF-β is lower.). Therefore, by comparing the concentration of diagnostic biomarkers in a biological sample collected from a subject, data is obtained to determine whether the subject is a mitochondrial disease patient or not. A kit to measure the diagnostic biomarker can be provided.

Moreover, a diagnostic kit, and a therapeutic kit comprising the diagnostic kit and a mitochondrial disease therapeutic agent are provided. They are preferably used.

Incidentally, the actual diagnosis is carried out by adding the comprehensive judgment by qualified personnel (e.g., a physician) based on the data obtained by the measuring method of the present invention.

### BRIEF DESCRIPTION OF THE FIGURES

FIG. 1 shows the results of comprehensive gene expression analysis of GDF15 expression levels. 2SA means control cells, 2SD means mitochondrial disease model cells, GDF15 expression levels were determined over time after treated with high concentration of lactate (Lactate) and pyruvate (Pyruvate).

FIG. 2 shows the results of the quantitative RT-PCR of GDF15 expression levels. The open bar indicates control cells (2SA), the black bar indicates the mitochondrial disease model cell (2SD), GDF15 expression level were determined over time after treated with high concentration of lactate (Lactate) and pyruvate (Pyruvate) (same in FIG. 3 and FIG. 4).

FIG. 3 shows the results of quantitative RT-PCR of INHBE expression levels.

FIG. 4 shows the results of quantitative RT-PCR of IL1A expression levels.

FIG. 5 shows the results of ELISA of GDF15 concentrations in the medium of cell cultures. 2SA means control cells, 2SD means mitochondrial disease model cells, GDF15 protein levels were determined in normal condition (1P), high concentration of lactate (10L and high concentration of pyruvate (10P).

FIG. 6 shows the results of measurements of cytokines concentrations in blood from mitochondrial diseases patients and controls. Mitochondrial disease patients (n=18) contain MELAS syndromes (n=15) and other mitochondrial diseases (n=3), control subjects (n=13) contain other pediatric disease patients. Cytokines are IL-16, IL-18, CTACK, HGF, MIF, MIG, SCF, SCGF-β and SDF-1α.

FIG. 7 shows the results of receiver operating characteristic curve analysis of biomarkers. (A) shows the results of ELISA of FGF21 in blood from mitochondrial disease patients (n=16) and control subjects (other pediatric disease patients (n=10)), (B) is a comparison of concentration correlation FGF21 and GDF15,(C) shows the receiver operating characteristic curve of GDF15, HGF, MIG, SCF, SCGF-β and FGF21, respectively.

FIG. 8 shows the results of ELISA of GDF15 (pg/mL) in serum. Control means control subjects, MtD means mitochondrial disease patients.

### MODES FOR CARRYING OUT THE INVENTION

Next, embodiments of the present invention will be described with reference to the drawings. The scope of the invention is not limited by these embodiments and can be embodied in various forms without changing the essentials of the invention.

In this study, for the purpose of diagnostic biomarkers research of mitochondrial diseases, establishment of the experimental system using mitochondrial disease model cells, identification of candidate biomarkers by exhaustive gene expression analysis and verification by clinical specimens were carried out.

1. Establishment of the Experimental System Using Mitochondrial Disease Model Cells

(i) Cybrid cells established from myoblasts in MELAS (mitochondrial myopathy, encephalopathy, lactic acidosis, and stroke-like episodes), which is relatively high incident among mitochondrial diseases patient and human osteosarcoma 143B cells were used in the experiment. Specifically, myoblast cells from MELAS patients lacking the cell nuclei were fused with human osteosarcoma-derived 143B cells lacking mitochondrial DNA (mtDNA). From some fused cell lines, a cell line without m.3243A>G mutation in mtDNA was used as control cell (2SA) and a cell line with m.3243A>G mutation with 94% was used as mitochondrial disease model cell (2SD) (the mutation ratio is not 100%, because mitochondria is heteroplasmy).

Cells were cultured in high-glucose Dulbecco's modified Eagle's medium (DMEM) supplemented with 10% FBS, 1 mM sodium pyruvate, and 0.4 mM uridine at 37° C. under a humidified atmosphere of 5% CO2.

(ii) Metabolome analysis were performed using 2SA cells and 2SD cells. The effect of pyruvate in the energy metabolism of mitochondrial disease model cell was revealed. The energy metabolism disorder has become remarkable 4 hours after treatment of high concentration of lactic acid (10 mM) in 2SD cells, and the phenomenon was not observed after treatment of high concentration of pyruvic acid (10 mM). The energy metabolism disorder was not observed after treatment of high concentration of lactic acid in 2SA cells. Based on the results, we came up with the forecast that the genes expressed in 2SD cells with energy metabolism disorders in a high concentration of lactic acid may be new biomarkers that reflects the energy metabolic disorder of mitochondrial disease patients. Therefore, we searched for genes significantly increased expression only when 2SD cells treated with 10 mM lactate with an exhaustive analysis of gene expression in 2SA cells and 2SD cells treated with 10 mM lactate or 10 mM pyruvate.

(iii) In order to examine the experimental conditions of comprehensive gene expression analysis, quantitative RT-PCR of 2SD cells cultured in the plurality of culture conditions were performed. The optimal experimental conditions were determined using gene expression levels which were suggested the amino acid starvation response genes CHOP and ASNS associated with mitochondrial dysfunction.

Detailed examination methods were as follows.

<Microarray Analysis>

Total RNA was isolated from cells by using a miRNeasy mini kit (Qiagen, Venlo, Netherlands). One hundred nanograms of total RNA was labeled and amplified with a low input quick amp labeling kit (Agilent Technologies, Santa Clara, Calif., USA) used according to the manufacturer's instructions. The labeled cRNA was hybridized to the Agilent SurePrint G3 Human GE 8×60K Microarray in a rotating hybridization oven at 10 rpm for 20 h at 65° C. After hybridization, the microarrays were washed according to the manufacturer's instructions and scanned on an Agilent DNA Microarray Scanner with Scan Control software. The resulting images were processed, and raw data were collected by using Agilent Feature Extraction software. Expression data were analyzed by using GeneSpring GX 11 (Agilent Technologies). The signal intensity of each probe was normalized by a percentile shift, in which each value was divided by the 75th percentile of all values in its array. For pairwise comparison analysis, only the probes that had expression flags present under at least one condition were considered. The list was analyzed with Ingenuity Pathways Analysis software (Ingenuity Systems, Redwood, Calif., USA).

<Quantitative RT-PCR>

Total RNA was isolated from cells with miRNeasy-minikit (Qiagen), and reverse transcribed to cDNA with a High Capacity cDNA Reverse Transcription Kit (Life Technologies, Carlsbad, Calif., USA) used according to the manufacturer's protocols. Real-time PCR was performed on the StepOnePlus Real-Time PCR System (Life Technologies) using Power SYBR Green PCR Master Mix. 18S rRNA gene was used as an internal control for normalization. The sequences of primers are listed in Table 1(The numbers in the sequence listing, SEQ ID NO: 1 to 1 stage left, SEQ ID NO: 2 to the right sequentially subjected to SEQ ID NO: 8.).

[Table 1]

TABLE 1
The sequence of primers for real-time PCR

| Gene | Forward Primer | Reverse primer |
|---|---|---|
| GDF15 | AGAGCTGGGAAGAT | CGAGAGATACGCAG |
| | TCGAACAC | GTGCAG |
| INHBE | GAGTGTGGCTCCAG | GGAGTGGACAGGTG |
| | GGAATG | AAAAGTGAG |
| IL1A | CTGCCCAAGATGAA | GTGAGTTTCCCAGA |
| | GACCAAC | AGAAGAGGAG |
| 18S rRNA | TCAACACGGGAAAC | CAGACAAATCGCTC |
| | CTCACC | CACCAAC |

<ELISA and Multiplex Suspension Array>

Cells were placed on 60-mm dishes 1 day before replacing the medium with fresh medium. Conditioned medium cultured for 24 h was collected, and the particulates were removed by centrifugation (at 500×g for 10 min, at 10,000×g for 30 min). The GDF15 and INHBE concentrations in the supernatants and in the sera of patients were determined in duplicate by using a Human GDF-15 Immunoassay (DGD150, R&D Systems, Minneapolis, Minn., USA) and enzyme-linked immunosorbent assay kit for Inhibin Beta E (E90048Hu, Uscn Life Science, Wuhan, Hubei, PRC) according to the manufacturer's instructions. For measuring IL1A and other cytokine concentrations, the sera were subjected to a multiplex suspension array, Bio Plex Pro Human Cytokine Grp II Panel 21-Plex (MF0-005KMII, Bio-Rad, Hercules, Calif., USA). The cytokines measured by use of this array were the following: IL-2Rα, IL-3, IL-12 (p40), IL-16, IL-18, CTACK, GRO-α, HGF, IFN-α2, LIF, MCP-3, M-CSF, MIF, MIG, β-NGF, SCF, SCGF-β, SDF-1α, TNF-β, and TRAIL.

<Statistical Analysis>

Statistical analyses were performed by using IBM SPSS statistics (IBM, Armonk, N.Y., USA). We used the nonparametric Mann-Whitney U test to validate differences in cytokine levels in serum between mitochondrial disease patients and controls. The correlation between GDF15 and FGF21 concentrations in serum was assessed by Spearman correlation analysis. We plotted the receiver operating characteristics (ROC) curve for GDF15, HGF, MIG, SCF, SCGF-βand FGF21 and calculated the area under the curve (AUC). The data for the sensitivity and 100 minus the specificity were plotted on a continuous scale.

2. Identification of Candidate Biomarkers By Exhaustive Gene Expression Analysis of Mitochondrial Disease Model Cell

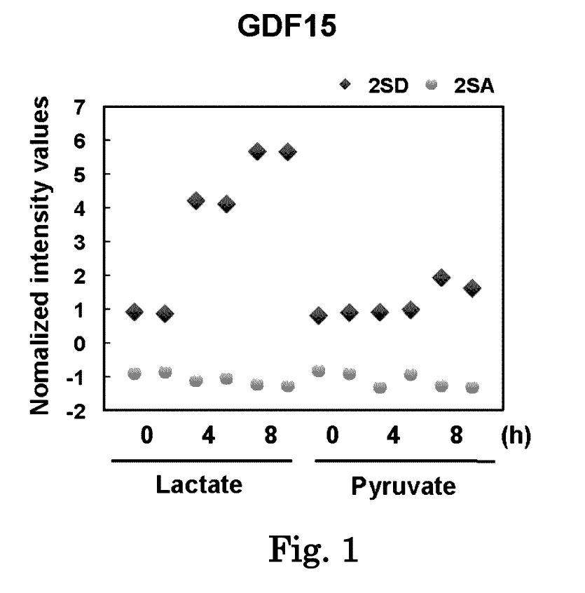

(i) 2SA cells and 2SD cells were collected at 0, 4, and 8 hours after treatment of 10 mM lactate or 10 mM of pyruvate. After RNA were extracted from these cells, exhaustive gene expression analysis were performed using microarray analysis. In the results, 313 genes significantly increased expression only when 2SD cells treated with 10 mM lactate were identified (FIG. 1).

(ii) To explore the measurable biomarkers in blood, 23 genes which encode secreted proteins from the 313 genes were identified (Table 2).

[Table 2]

TABLE 2
The genes specifically up-regulated by lactate treatment for 8 h

| | | | Fold Change | |
|---|---|---|---|---|
| Gene Symbol | Accession Number | Entrez Gene Name | L-8/L-0*¹ | L-8/P-8*² |
| GDF15 | NM_004864 | growth differentiation factor 15 | 27.4 | 14.8 |
| INHBE | NM_031479 | inhibin, beta E | 15.0 | 9.4 |
| AREG | NM_001657 | amphireguiln | 14.0 | 2.2 |
| ECM2 | NM_001393 | extracellular matrix protein 2, female organ and | 11.8 | 9.0 |
| | | adipocyte specific | | |
| ADM2 | NM_024866 | adrenomedullin 2 | 10.3 | 3.0 |
| MMP3 | NM_002442 | matrix metallopeptidase 3 (stromelysin 1, | 9.8 | 4.2 |
| | | progelatinase) | | |
| IL1A | NM_000575 | interleukin 1, alpha | 7.6 | 6.0 |
| C12orf39 | ENST00000256969 | chromosome 12 open reading frame 39 | 6.3 | 6.7 |
| APOL6 | NM_030641 | apolipoprotein L, 6 | 6.2 | 3.8 |
| SCG5 | NM_003020 | secretogranin V (7B2 protein) | 5.2 | 3.0 |
| SPOCK2 | NM_014767 | spare/osteonectin, cwcv and kazal-like domains | 5.1 | 6.6 |
| | | proteoglycan (testican) 2 | | |
| AMTN | NM_212557 | amelotin | 5.0 | 3.9 |
| IL23A | NM_016584 | interleukin 23, alpha subunit p19 | 4.4 | 2.8 |
| ADAMTS17 | NM_139057 | ADAM metallopeptidase with thrombospondin | 3.5 | 2.2 |
| | | type 1 motif, 17 | | |
| VEGFA | NM_001025370 | vascular endothelial growth factor A | 3.4 | 2.5 |
| STC2 | NM_003714 | stanniocalcin 2 | 3.4 | 2.6 |
| PDGFB | NM_002608 | platelet-derived growth factor beta polypeptide | 2.8 | 3.8 |
| C1QTNF1 | NM_198594 | C1q and tumor necrosis factor related protein 1 | 2.6 | 2.9 |
| HECW2 | NM_020760 | HECT, C2 and WW domain containing E3 | 2.4 | 2.1 |
| | | ubiquitin protein ligase 2 | | |
| IGFALS | NM_004970 | insulin-like growth factor binding protein, acid | 2.3 | 2.5 |
| | | labile subunit | | |
| IGFBPI | NM_000596 | insulin-like growth factor binding protein I | 2.3 | 2.1 |
| PDGFA | NM_002607 | platelet-derived growth factor alpha | 2.2 | 2.2 |
| | | polypeptide | | |
| CLEC3B | NM_003278 | C-type lectin domain family 3, member B | 2.1 | 2.2 |

*¹Fold change between 8 h and 0 h after lactate treatment
*²fold change between lactate treatment and pyruvate treatment at 8 h

In particular, six genes, i.e. GDF15, AREG, INHBE, ADM2, ECM2 and ILIA from 23 genes in table 2 showed significantly increased expression when treated with lactate. Four genes that showed decreased expression only when treated with 10 mM lactate in 2SD cells were identified (Table 3).

[Table 3]

TABLE 3
The genes specifically down-regulated in 2SD cells with lactate treatment for 8 h

| | | | Fold Change | |
|---|---|---|---|---|
| Gene Symbol | Accession Number | Entrez Gene Name | L-8/L-0*¹ | L-8/P-8*² |
| CXCL1 | NM_001511 | chemokine (C—X—C motif) ligand 1 | −3.4 | −2.6 |
| | | (melanoma growth stimulating activity, alpha) | | |
| PDZRN3 | NM_015009 | PDZ domain containing ring finger 3 | −2.4 | −2.0 |
| SLC39A10 | NM_020342 | solute carrier family 39 (zinc transporter), | −2.3 | −2.9 |
| | | member 10 | | |
| DKK1 | NM_012242 | dickkopf 1 homolog (Xenopus laevis) | −2.1 | −2.3 |

*¹Fold change between 8 h and 0 h after lactate treatment
*²Fold change between lactate treatment and pyruvate treatment at 8 h

(iii) Searching the documents on the genes whose expression was increased, three genes that are considered to have a high association with mitochondrial dysfunction (GDF15, inhibin beta E (INHBE), and interleukin-1α (ILIA)) were selected.

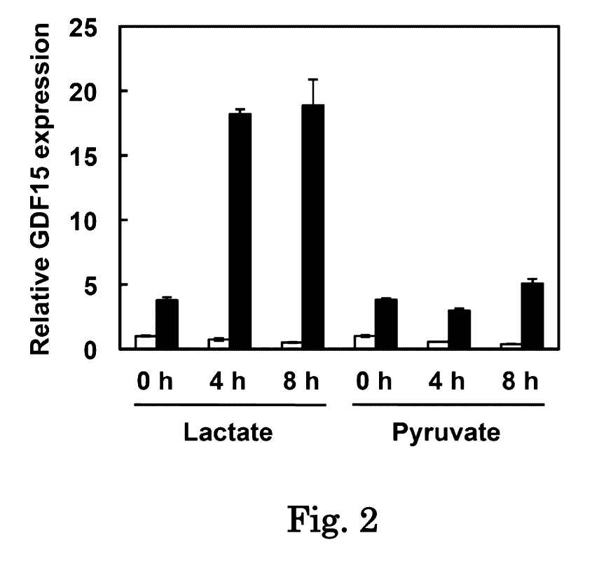

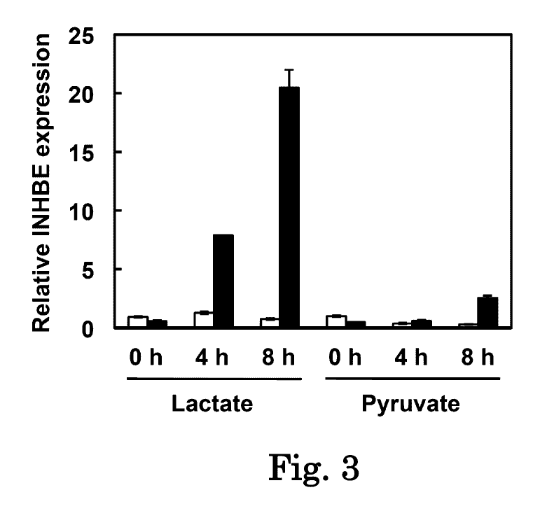

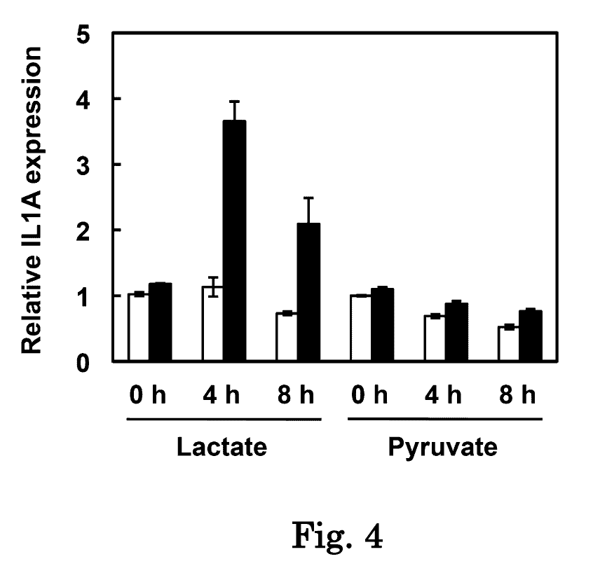

(iv) In order to determine the expression level of GDF15, INHBE and IL1A in cells, quantitative RT-PCRs were performed. The expression level of GDF15, INHBE, and IL1A were increased at 4 and 8 hours after treatment with 10 mM lactate in 2SD cells, otherwise they were not changed after treatment with 10mM pyruvate. The expression level of GDF15 was higher in 2SD cells than in 2SA cells at 0 hour. On the results, the reproducibility of the microarray data were confirmed. GDF15, INHBE, and IL1A were identified as biomarkers for mitochondrial diseases (FIG. 2 to FIG. 4).

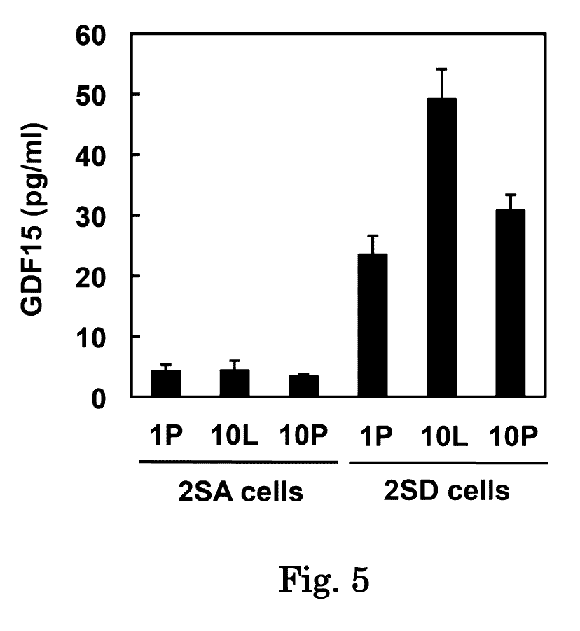

(v) The concentrations of three secreted proteins as candidate biomarkers in cell culture were measured by ELISA and multiplex suspension arrays. As a result, the concentration of GDF15 (growth differentiation factor 15) in culture medium of 2SD cells was higher than that in culture medium of 2SA cells under normal culture conditions (1 mM pyruvate administration). Further, the concentration of GDF15 in culture medium increased by treatment of 10 mM lactate in 2SD cells (FIG. 5). Other secreted proteins could not be measured under the detection limit.

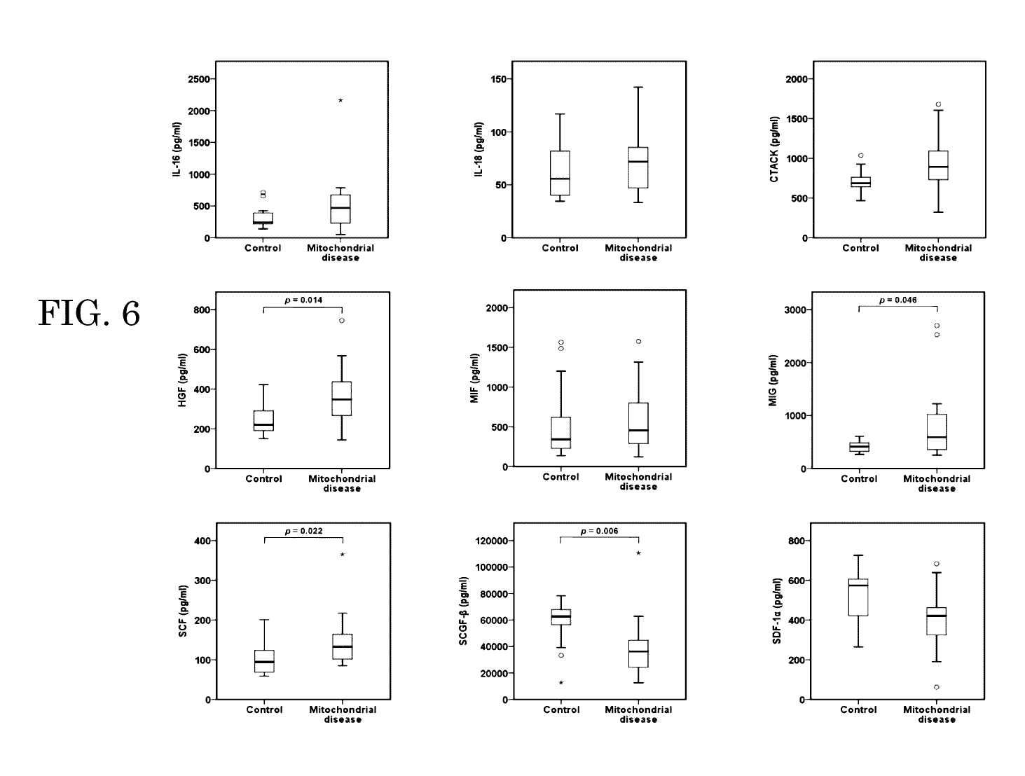

(vi) As shown in FIG. 6, the concentrations of other proteins, hepatocyte growth factor in blood (HGF), gamma interferon induced monokine (MIG), and SCF in mitochondrial disease patients were significantly higher than those in control subjects. Also, the concentration of SCGF-βin mitochondrial disease patients was significantly lower than that in control subjects. Therefore, these four proteins were determined to be useful as diagnostic markers for mitochondrial diseases.

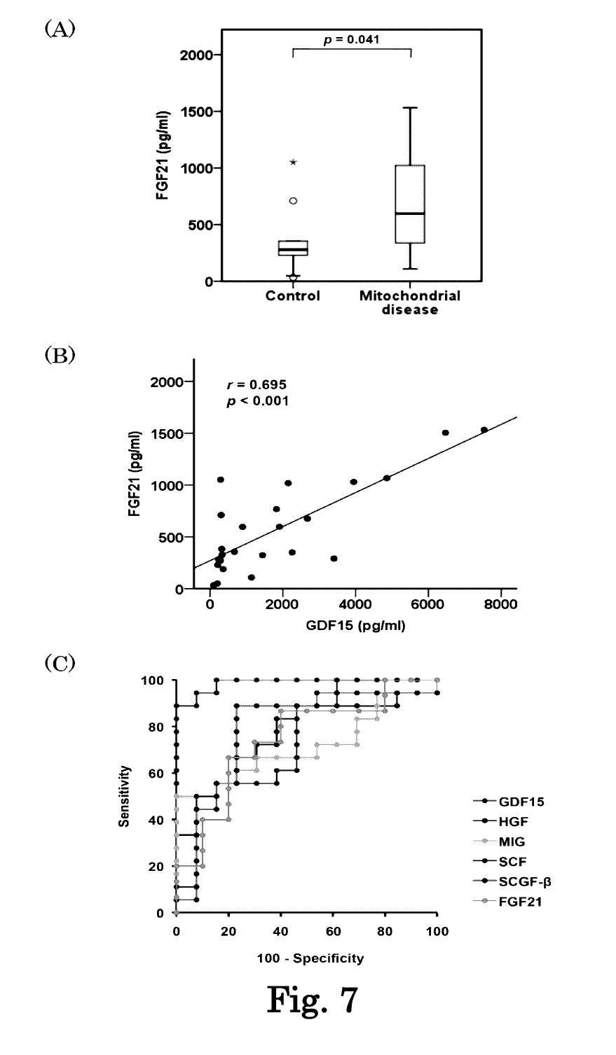

The concentration of serum FGF21 in mitochondrial disease patients was higher than that in control subjects (FIG. 7(A)). FGF21 had been pointed out the relationship with mitochondrial diseases. The blood levels of FGF21 in patients had been known higher than that in normal subjects. In the study, FGF21 concentrations in mitochondrial disease were higher than those in other disease patients. Further, GDF15 concentrations in blood showed good correlation with the concentrations of FGF21 (FIG. 7 (B)). Receiver operating characteristic curve (ROC curve) of HGF, MIG, SCF, SCGF-β, FGF21, and GDF15 were created. As a result, all proteins were associated with the disease, as shown in FIG. 7(C). For the diagnosis of mitochondrial disease, GDF15 was superior to other proteins with most sensitivity and specificity. Comparing the area under the curve (AUC), GDF15 was 0.987, it was higher than any other proteins, HGF (0.761), MIG (0.714), SCF (0.744), SCGF-β (0.791) and FGF21 (0.763).

3. Verification of Candidate Biomarkers By Clinical Specimens

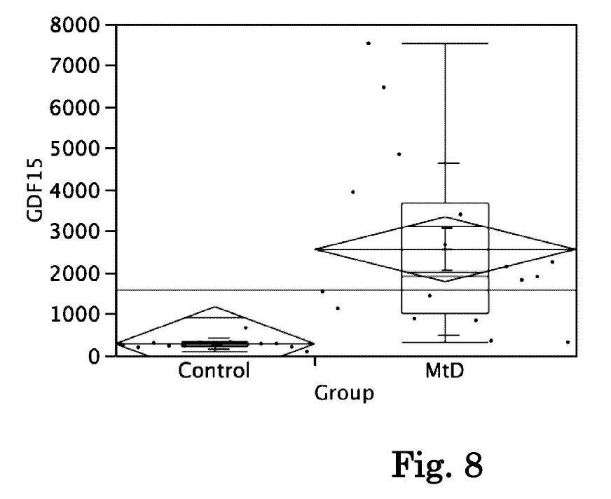

Finally, the concentrations of GDF15 in blood were examined in mitochondrial disease patients and other pediatric diseases patient by ELISA. GDF15 concentrations were significantly increased in mitochondrial disease patients (FIG. 8). The concentrations of other two secretory proteins could not be determined under the detection limit.

From these results, GDF15 is used as new mitochondrial disease marker and a marker of mitochondrial dysfunction.

4. Study of sodium pyruvate therapy for mitochondrial disease patients For mitochondrial disease patients (n=10), sodium pyruvate (0.3 g/kg to 2 g/kg) had been administered for over seven years. During the period, the transition of

FGF-21 and GDF-15 had been determined and parameters of other therapeutic effects had been investigated. The mitochondrial disease patients contained PDH E1A deficiency, MELAS/cardiomyopathy with A3243G mutation, and MELAS/Leigh syndrome with G13513A mutation in the ND5 gene.

The concentrations of lactate, pyruvate, alanine, and GDF-15 were significantly reduced with sodium pyruvate therapy. Moreover, no side effects were observed. Therefore, sodium pyruvate therapy for mitochondrial disease patients was highly effective therapy.

According to the embodiments, GDF15, HGF, MIG, SCF and SCGF-β were used for diagnostic biomarkers of mitochondrial disease. The blood levels of GDF15, HGF, MIG, SCF and SCGF-β in the mitochondrial disease patients are different from those in the control subjects (the levels of GDF15, HGF, MIG and SCF is higher and the level of SCGF-β is lower.). Therefore, by comparing the concentration of diagnostic biomarkers in a biological sample collected from a subject, a data is obtained to determine whether the subject is a mitochondrial disease patient or not. A kit to measure the diagnostic biomarker can be provided.

Moreover, a diagnostic kit, and a therapeutic kit comprising the diagnostic kit and a mitochondrial disease therapeutic agent were provided. They were preferably used.

Incidentally, the actual diagnosis is carried out by adding the comprehensive judgment by qualified personnel (e.g., a physician) based on the data obtained by the measuring method of the present invention.

1. A measuring method that obtains data associated with a mitochondrial disease comprising:
measuring the level of at least one protein selected from the group consisting of GDF15 (growth differentiation factor 15), HGF (hepatocyte growth factor), MIG (gamma interferon induction monokine), SCF (stem cell factor) and SCGF-β (stem cell growth factor beta) in a biological sample collected from a subject;
comparing the protein level to that of control subjects; and
checking whether or not there is difference between the protein level of the subject and that of control subjects.

2. The method according to claim 1, wherein checking the level of GDF15, HGF, MIG and SCF in the biological sample collected from the subject is higher than that in the biological sample collected from control subjects; and checking the level of SCGF-β in the biological sample collected from the subject is lower than that in the biological sample collected from control subjects.

3. measuring method that obtains data associated with a mitochondrial disease comprising:
measuring the level of mRNA of at least one protein selected from the group consisting of GDF15 (growth differentiation factor 15), HGF (hepatocyte growth factor), MIG (gamma interferon induction monokine), SCF (stem cell factor) and SCGF-β (stem cell growth factor beta) in a biological sample collected from a subject;
comparing the mRNA level to that of control subjects; and
checking whether or not there is difference between the mRNA level of the subject and that of control subjects.

4. The method according to claim 1, wherein the biological sample is blood.

5. A measurement kit for carrying out the method according to claim 1, comprising an antibody that specifically recognizes a protein selected from the group consisting of GDF15, MIG, SCF and SCGF-β.

6. A measurement kit for carrying out the method according to claim 3, comprising DNA that recognizes mRNA that expresses the protein selected from the group consisting of GDF15, HGF, MIG, SCF and SCGF-β.

7. (canceled)

8. (canceled)

9. A therapeutic kit comprising:
the kit according to claim 5 and
a therapeutic agent for a mitochondrial disease.

10. The therapeutic kit according to claim 9, wherein the therapeutic agent for a mitochondrial disease is selected from the group consisting of sodium pyruvate, coenzyme Q10 or coenzyme Q10 analogue, EPI-743 and L-arginine.

11. The method according to claim 2, wherein the biological sample is blood.

12. A measurement kit for carrying out the method according to claim 2, comprising an antibody that specifically recognizes a protein selected from the group consisting of GDF15, HGF, MTG, SCF and SCGF-β.

13. The method according to claim 3, wherein the biological sample is blood.

14. (canceled)

15. A measurement kit for carrying out the method according to claim 4, comprising DNA that recognizes mRNA that expresses the protein selected from the group consisting of GDF15, HGF MIG, SCF and SCGF-β.


SEQUENCE LISTING (8 sequences)

SEQ ID NO: 1 - DNA, 22 bp, Homo sapiens
agagctgggaagattcgaacac

SEQ ID NO: 2 - DNA, 20 bp, Homo sapiens
cgagagatacgcaggtgcag

SEQ ID NO: 3 - DNA, 20 bp, Homo sapiens
gagtgtggctccagggaatg

SEQ ID NO: 4 - DNA, 23 bp, Homo sapiens
ggagtggacaggtgaaaagtgag

SEQ ID NO: 5 - DNA, 21 bp, Homo sapiens
ctgcccaagatgaagaccaac

SEQ ID NO: 6 - DNA, 24 bp, Homo sapiens
gtgagtttcccagaagaagaggag

SEQ ID NO: 7 - DNA, 20 bp, Homo sapiens
tcaacacgggaaacctcacc

SEQ ID NO: 8 - DNA, 21 bp, Homo sapiens
cagacaaatcgctccaccaac